(19) United States
(12) Patent Application Publication
(10) Pub. No.: US 2007/0144907 A1
(43) Pub. Date: Jun. 28, 2007

(54) Integrated two-dimensional gel electrophoresis

(75) Inventors: Martin Kopp, Rotkreuz (CH); Roger Sandoz, Rotkreuz (CH); Mario Curcio, Rotkreuz (CH); Michael Glauser, Rotkreuz (CH)

Correspondence Address: DINSMORE & SHOHL, LLP;ONE DAYTON CENTRE, ONE SOUTH MAIN STREET, SUITE 1300, DAYTON, OH, 45402, US

(73) Assignee: Roche Diagnostics Operations, Inc., Indianapolis, IN (US)

(21) Appl. No.: 11/643,729

(22) Filed: Dec. 21, 2006

(30) Foreign Application Priority Data
Dec. 28, 2005 (EP) EP 05028551.9

(51) Int. Cl. C07K 1/26; G01N 27/00

(52) U.S. Cl. 204/456; 204/606

(57) ABSTRACT

A disposable electrophoresis device for separation of a complex protein sample, using two-dimensional gel electrophoresis and method thereof are disclosed. The device comprises a carrier with a first area for a first dimension gel strip and a second area for a second dimension gel, where the two areas are directly in contact with each other. The device further comprises a body made of a hard component and an elastic component layer at a variable distance from the carrier. The body further comprises at least one cavity for external actuation of at least one valve, wherein the valve is represented by the elastic component layer.

### RELATED APPLICATIONS

This application claims priority to EP 05028551.9 filed Dec. 28, 2005.

### FIELD OF THE INVENTION

The present invention refers generally to two-dimensional gel electrophoresis, and in particular to disposable electrophoresis device for the separation of a complex protein sample using two-dimensional gel electrophoresis and a method thereof.

### BACKGROUND OF THE INVENTION

Two-dimensional slab gel electrophoresis is still the most used approach to proteomics and it might be still for several years, despite alternative chromatographic methods are gaining popularity, if after improvement over the years, other limitations still present are addressed. In particular, this remains a time-consuming and laborious procedure, requiring trained personnel, on the hands of whom the quality of results is mainly depending. Just because there is much manual work involved, reproducibility is indeed difficult to achieve, whereas on the other hand gels are mostly made to be compared. Although running conditions can be quite reproducible, as these are controlled by proper set-up and power supplies, and new buffer systems have increased gel stability and performance, problems with accuracy and consistency can arise from variations in the other numerous parameters to keep under control. Some of these are for example, sample loading and rehydration, in terms of sample amount, losses, and homogeneity of the strip, strip handling with risk of damaging and contamination, imprecise and slow coupling of the strip to the gel, gel casting and polymerization, in terms of homogeneity, casting and reaction speed, especially for gradients, air sensitivity, time for completion until run is started, risk to trap bubbles causing consequently also field discontinuities, increase in temperature during the run, pH and viscosity changes, and loss of buffer capacity.

Describing in detail the entire gel electrophoresis process is out of the scope of this invention as several reviews can be retrieved in the literature. A brief overview is however given below to help in understanding.

Normally, first dimension separation consists of isoelectric focusing (IEF) where proteins separate according to their isoelectric point in a pH gradient, typically immobilized (IPG), in a long and narrow supported gel assuming the form and taking the name of a gel strip. The gel strips, commercially available, are normally supplied in a semi-dry state and they have to be rehydrated with the sample solution before analysis. This operation takes from a few hours to typically overnight and usually takes place under mineral oil to prevent drying and crystallization of urea present in the sample solution. IEF takes place also under mineral oil for the same reasons in the same or a different tray with the gel strip in contact with two electrodes at the sides, between which a high voltage is applied.

After IEF the gel strip has to be equilibrated, which means that the proteins focused within the gel strip have to be first alkylated and then complexed with sodium dodecyl sulfate (SDS) in order to be later transferred to a second dimension gel wherein the proteins are separated according to size. Reduction/Alkylation can be achieved by different reagents and one has the option to perform this step during sample preparation before rehydration such as suggested by Herbert et al. (Herbert B et al. (2001). Reduction and alkylation of proteins in preparation of two-dimensional map analysis: why, when and how? Electrophoresis 22, 2046-2057), although this might result in shifts of the isoelectric points. However SDS equilibration can be performed only after IEF, so that the gel strip is literally washed for several minutes in the equilibration solution containing SDS. The gel strip is then placed on top and in contact with a prepolymerized SDS polyacrylamide gel and coupling is achieved by pouring a hot agarose solution over the strip. This is usually accomplished between two glass plates which are clamped together and then placed in a buffer containing cassette where voltage is applied across the gel. The gel might be formed with a porosity gradient in order to increase resolution in the second dimension. After this is complete, the gel is removed, fixed, stained and background staining dye removed before proceeding eventually with the subsequent steps, i.e. spot picking, digesting, and mass spectrometry analysis.

Ideally, what is desirable is that no further manual intervention is required after the sample has been loaded, in a way similar to the instrumental chromatographic approach, the main strength of which is indeed automation associated with better reproducibility. Automation and integration of the steps involved in the gel-based procedure is a challenge that others in the art are also addressing.

An integrated, fully automated system, mimicking step by step the manual procedure, including also sample preparation and gel strip casting is described in U.S. Pat. No. 6,554,991 B 1. The robotic machinery behind it, the complexity of the operation and the investment necessary go however far beyond a practical and widespread use of it, especially among the smaller research laboratories.

Published US patent application No. 2003/0127331 discloses a system where the gel strip once cast at the bottom of a vertical mold formed by two plates doesn't have to be moved after IEF. It is understood that the gel strip can be treated with the equilibration solution, apparently just from one side, and subsequently coupled to the second dimension gel by pouring the gel solution into the mold directly in contact with the gel strip or on top of an agarose layer. Doubts remain however concerning the efficiency and/or the time of the equilibration with the SDS having to diffuse inside the gel strip just from one side and whether the resolution obtained in the first dimension can be preserved. As no mention is made concerning the polymerization method, the long times associated with the classical method increase further the concern about loss of resolution. Also, the way the gel strip is formed and the sample is added is less reproducible and the fact that a sealing tab at the bottom of the mold has to be removed at the end is not practical.

In EP 0366897 it is disclosed that the gel strip is first separated from a prepolymerized gel by means of a non-conductive phase-change material, which is melted after IEF by increasing the temperature, removed and substituted with other gel medium. No reference is however made to the equilibration step and besides the concerns about the effect of the temperature for proteins and gel, remains the problem associated with closing and opening this time the top of the mold.

Other barrier means between a gel strip and gel are disclosed in WO 02/084273 A1. The first embodiment reported therein making use of sliding solid barriers is certainly not the most advantageous as formed gels might be disrupted by this action. A more interesting solution makes use of pneumatically assisted valves consisting of soft and expandable material, separating the strip from a preferably precast gel. The space occupied by the valve is later filled with agarose for coupling. In a third embodiment, semi-walls at the sides of the gel strip, are used as gasket against which a foil used also as gel support can be pressed, thus opening and closing the strip chamber by changing position relative to the opposite rigid surface. The gel solution is in this case introduced and polymerized preferably after opening the strip chamber at the end of the first dimension or, with difficulty to imagine as the foil has to move, the gel can be precast. Although the possibility to immerse the gel strip with the gel solution in one step is disclosed, the use of agarose is again preferred. Equilibration solutions can access the gel strip through the rigid part of the device.

There are, however, weak points still left in the above disclosed system, first of which is represented by dead volumes for the sample regardless of the embodiment. Indeed, an extra space is necessary above the fully rehydrated gel strip in order to allow the flow of the equilibration solution after the first dimension, thus requiring excess of sample to fill that same gap when rehydrating the gel strip. Also, the fact that excess liquid is left above the gel strip during IEF can result in disturbed focusing and horizontal striking, and if removed can result in drying of the gel strip. Moreover, loss of resolution due to the long waiting time for gel polymerization and deleterious effects due to penetration of acrylamide monomers into the gel strip can be expected when casting the gel after IEF, as no methods of polymerizing the gel solution, other than the intended classical one, are claimed or even mentioned. This must be the reason why the use of agarose is preferred in all cases. Agarose brings however new annoying issues, like the need to be boiled before melting and the troubles to remove it from all the tubing and fittings once it has started to gel. Concerns remain also regarding the efficacy in maintaining an even and non deformed foil shape, important in guaranteeing a homogeneous gel thickness.

### SUMMARY OF THE INVENTION

It is against the above background that the present invention provides an improved disposable device and ways of processing the same. The present invention provides convenient and effective means of integrating the generally accepted and manually executed steps for the separation of a complex protein sample in proteomics analysis based on two-dimensional gel electrophoresis. In particular, the present invention provides a system having a disposable electrophoresis device for sample separation based on two-dimensional electrophoresis, which requires only minimal or preferably no manual intervention once the sample has been loaded to the device.

In one embodiment, a disposable electrophoresis device for separation of a complex protein sample using two-dimensional gel electrophoresis is disclosed. The device comprises a carrier with a first area for a first dimension gel strip and a second area for a second dimension gel where the two areas are directly in contact with each other; and a body made of a hard component and a flexible component layer, the body being provided at a variable distance from the carrier, the body defining at least one cavity used for external actuation of at least one valve, wherein closing of the valve is represented by a reversible stretching of the flexible component layer in a direction towards the carrier upon external actuation of the valve through the cavity.

These and other features and advantages of the present invention will be more fully understood from the following description of various embodiments of the present invention taken together with the accompanying drawings.

### BRIEF DESCRIPTION OF THE DRAWINGS

The following detailed description of the embodiments of the present invention can be best understood when read in conjunction with the following drawings, where like structure is indicated with like reference numerals and in which:

FIGS. 1a and 1b show schematically and not to scale, embodiments according to the present invention having a two-component disposable core part with closed valves and two variable thicknesses, one during rehydration and IEF (FIG. 1a) and the second thickness during equilibration (FIG. 1b).

FIGS. 2a and 2b show schematically and not to scale, two embodiments according to the present invention for a two component disposable core part during gel-casting in the second dimension.

FIGS. 3a and 3b are flow diagrams of a process for sample separation based on two-dimensional electrophoresis according to the present invention.

FIG. 4 is a diagram of an embodiment showing slits or cavities at a gel/buffer interface sealed by an appropriate membrane or tape according to the present invention.

FIG. 5 is a diagram showing one embodiment of creating slits or cavities at a gel-/buffer-interface by means of a blade function integrated in a processing instrument after gel-casting and polymerisation according to the present invention.

FIG. 6 is a diagram showing schematically one embodiment in which external buffer reservoirs for the second dimension are not integrated in a disposable electrophoresis device according to the present invention but are clamped by instruments when needed, while buffer is freshly circulated from larger reservoirs upstream in order to prevent the loss of buffer capacity associated with small reservoirs and small buffer volumes.

Skilled artisans appreciate that elements in the figures are illustrated for simplicity and clarity and have not necessarily been drawn to scale. For example, the dimensions of some of the elements in the figures may be exaggerated relative to other elements to help improve understanding of the embodiment(s) of the present invention.

### DETAILED DESCRIPTION OF THE INVENTION

Below and with reference to the following schematic drawings a brief description of examples of systems and processes according to the present invention are disclosed.

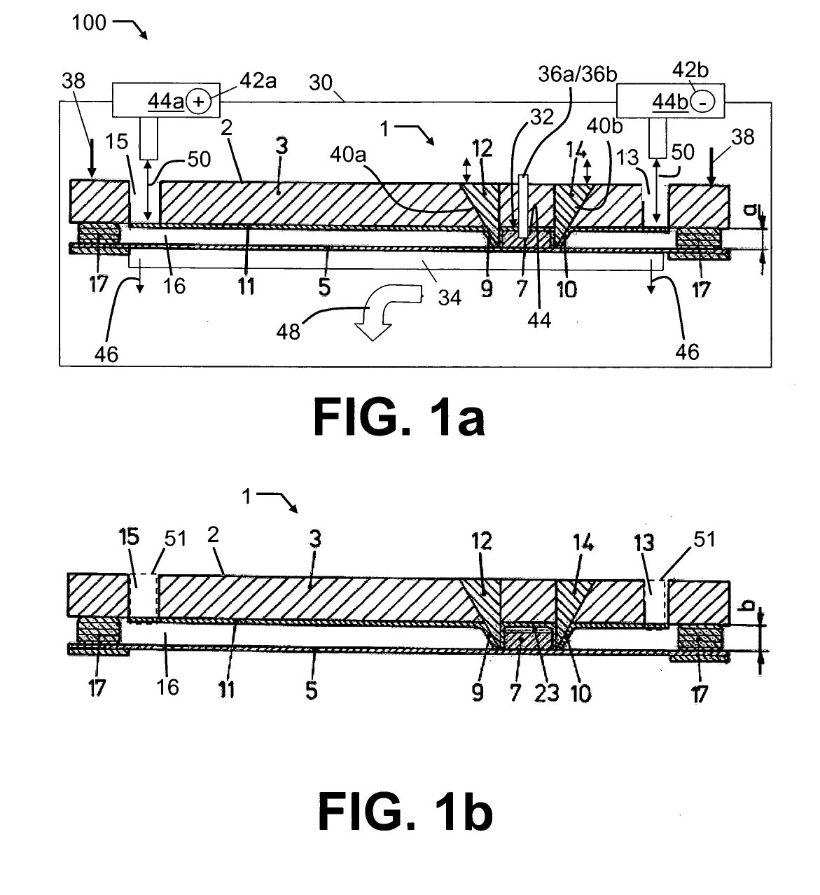

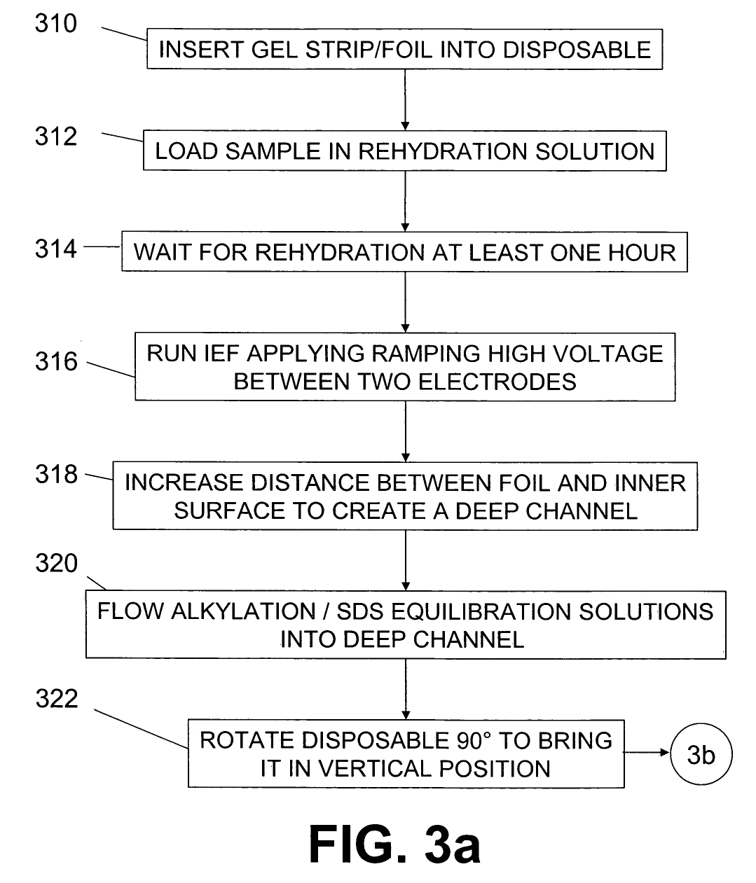

A two-dimensional electrophoresis system, generally indicated by reference symbol 100 in FIG. 1a, comprises a disposable electrophoresis device 1 which is subjected to a series of automatic operative steps, such as for example, illustrated by FIGS. 3a and 3b, by a e.g. software-controlled instrument 30, elements of which are also part of the invention in combination with the process, the disposable format and its function. With the terms “instrument” and “processing instrument” as used herein, an arrangement comprising e.g. buffer reservoirs, a cooling block, electrodes, tubing, UV lamps, etc. for processing the disposable electrophoresis device is understood.

A body 2 of the electrophoresis device 1, which includes at least one valve (e.g., valves 9 and 10), is injection molded by e.g. applying component molding technology, meaning that no assembly is needed between the parts and thus reducing the cost of manufacturing. In one embodiment, a generally rigid, inflexible or hard component 3 of the body 2 is a material chosen among acrylic glass (i.e. polymethylmethacrylate (PMMA)), polycarbonate (PC), polyethylene (PE), polypropylene (PP), polyethylene terephthalate (PET or PETE), or cyclo-olefin copolymers (COC) or any other suitable polymer material which can be molded and that is UV transparent, with the specific intent to allow UV-initiated fast polymerization of the gel solution during the process by shining light of appropriate wavelength directly through it, from one or multiple lamps integrated into the instrument 30. Another component of the body 2 is a first soft, flexible, or elastic component layer 11. In one embodiment, the first elastic component layer 11, which portions thereof defines valves 9 and 10 of the device 1, is a material chosen among thermoplastic elastomers (TPE) that is compatible with the previously chosen body material 3 such as, for example, PTS—Thermoflex® (Plastic Technology Service Ltd, Salisburg SP5 4BZ, UK), Santoprene® (Advanced Elastomer Systems, L.P., Akron, Ohio, USA), or any other suitable elastic material that can be molded in a convenient flat laminar shape and be directly attached to an inner surface of the hard component 3 of the disposable body 2 and to which a second dimension gel (discussed hereafter in a later section) does not stick. In one embodiment, in addition to the above requirements, the first elastic component layer 11 may further be a UV transparent material. By defining two slits or cavities 40a and 40b through the disposable body 2 immediately flanking a first zone provided for a gel strip laying underneath, the first elastic component layer 11 can be stretched down by external rigid actuation performed by the instrument 30, such as via inserts 12 and 14 of the instrument 30, thus trapping a gel strip 7 in a closed and tight environment (i.e., a strip chamber 32) by pressing against a gel strip carrier 5. The carrier 5 is a material selected from a polymer film, a foil, or a glass plate. In one embodiment, the carrier 5 is provided with a gel-bond material such that the second dimension gel can be firmly attached thereon. Similarly, in other embodiments, slits or cavities can be designed also in other positions along the hard component 3 of the body 2 to divide a gel chamber 16 defined between the carrier 5 and the first elastic component layer 11 of the device 1 into compartments if needed or simply to close any eventual open edge.

A second elastic component 17 is also integrated in the disposable electrophoresis device 1 as a gasket or ring provided along the perimeter of the area defining the gel chamber 16 (i.e., a second area for the second dimension gel). The second elastic component 17 is provided in sandwich arrangement between the inner surface of the body 2 and the carrier 5. In one embodiment, the second elastic component 17 is molded at the same time and with the same procedure, and also with the same material as the first elastic component layer 11. The second elastic component 17 is provided for the function of allowing variation (i.e., increasing and decreasing) of the distance between the two opposite surfaces of the first elastic component layer 11 and the carrier 5 upon external applied pressure 38, such as provided by the processing instrument 30. In one embodiment, the distance between the two opposite surfaces of the first elastic component layer 11 and the carrier 5 corresponds to about 0.7 mm during rehydration and IEF, which is indicated by symbol a in FIG. 1a, and to about 1.0 mm during equilibration, gel casting, polymerization, and second dimension separation, which is indicated by symbol b in FIG. 1b.

In one embodiment, the carrier 5, which cross-links to the gel during polymerization, is laminated to the disposable device 1 and can be peeled off by the user after the process.

In addition, in one embodiment, the outer dimensions of the disposable device 1 are designed according to an industry standard in order to facilitate robotic handling, and in one particular embodiment is according to the ANSI SBS (American National Standards Institute, The Society of Biomolecular Screening) standard with dimensions of: 127.76±0.25 mm×85.48±0.25 mm.

In one embodiment, disposable electrodes (not shown) consisting of an inexpensive material, such as for example, graphite, conductive paper, etc., can be integrated in the disposable device 1 for the first dimension separation. In the illustrated embodiment of FIG. 1a, electrode rods 36a and 36b (same on side not shown) made of a conductive metal such as, for example, copper or platinum, and which are part of the instrument 30, are made to contact the gel strip 7 at the extremities through holes or ports in the disposable body 2. In other embodiments, other such holes or ports are provided in the body 2 for sample loading, flowing of the equilibration solutions, casting of a gel solution for forming the second dimension gel, and venting. In one embodiment, integrated into the instrument 30 are the electrodes 42a and 42b for the second dimension, which are inserted into buffer reservoirs of the disposable at the appropriate time. Another embodiment has at least one inexpensive electrode (not shown) integrated in the disposable, for example, at the cathode, where redox reactions and electrode consumption are less important.

In another embodiment, in order to make the disposable device 1 even simpler and more compact, buffer reservoirs 44a and 44b for the second dimension are part of the instrument 30 and clamped to the disposable when needed by simple means making use of a gasket (not shown) and external pressure. In order to prevent the loss of buffer capacity associated with small reservoirs and small buffer volumes, in one embodiment the buffer is freshly circulated from larger reservoirs (not shown) upstream. In order to prevent keeping the entire circuit under the applied voltage during buffer replacement, in one embodiment, a “stop and go” discontinuous approach by opening the circuit defined between the electrodes 42a and 42b at intervals to replace the buffer is used In another embodiment, buffer replacement is achieved by restricting the channel of communication between large and small reservoirs, eventually also dispensing air bubbles along the liquid path as insulators. In an alternative embodiment, the limitation of the buffer capacity is overcome by recirculating the cathode buffer with the anode buffer and vice versa. By this means, the buffer reservoirs 44a and 44b can be kept small with no additional need of buffer during the run.

In order to maintain the flatness and evenness of the carrier 5, important especially to guarantee gel homogeneity and efficient cooling, both fundamental for reproducibility, a cooling block 34 of the instrument 30, on which the disposable device 1 geometrically fits, such as for example, via the external side of the carrier 5 contacting thereon, is provided. The cooling block 34 in one embodiment is made of porous ceramics, and in other embodiments is another porous or holes containing material such as metal, other heat-conductive alloy, polymers, and combinations thereof through which a vacuum suction, indicated by reference symbol 46, can be applied. At the same time this represents an advantageous way to steadily fix the disposable device 2 into the instrument 30 so that in one embodiment it is rotated about 90° during gel casting by mechanical rotation, indicated by reference symbol 48, of the cooling block 34.

In one embodiment, the disposable device 1 is generic containing no IEF strip, i.e., gel strip 7, thus leaving the freedom to the operator to insert a desired strip with a desired pH range, and avoiding the need to deliver and store the entire disposable device 1 with the strip inside at refrigeration temperature. Guiding features are provided so that no misplacing can occur, e.g. with closed valves, while the electrodes 36a and 36b in one embodiment serve also to keep the gel strip 7 in place. In another embodiment, with the disposable device 1 made simpler and compact as described above, the gel strip 7 is integrated into the disposable device 1, so that one could order different sets of disposables containing different strips. In still another embodiment, the gel strip 7 is already attached on the carrier 5, with the carrier 5 being delivered separately from the disposable body 2. In such an embodiment, the user then has to assemble the two parts together, e.g. with the help of positioning holes. In still yet another embodiment, the gel strip 7 is inserted through an opening in the carrier 5, which is provided as a bottom cover to the body 2, which is then closed afterwards with a tape-like mechanism.

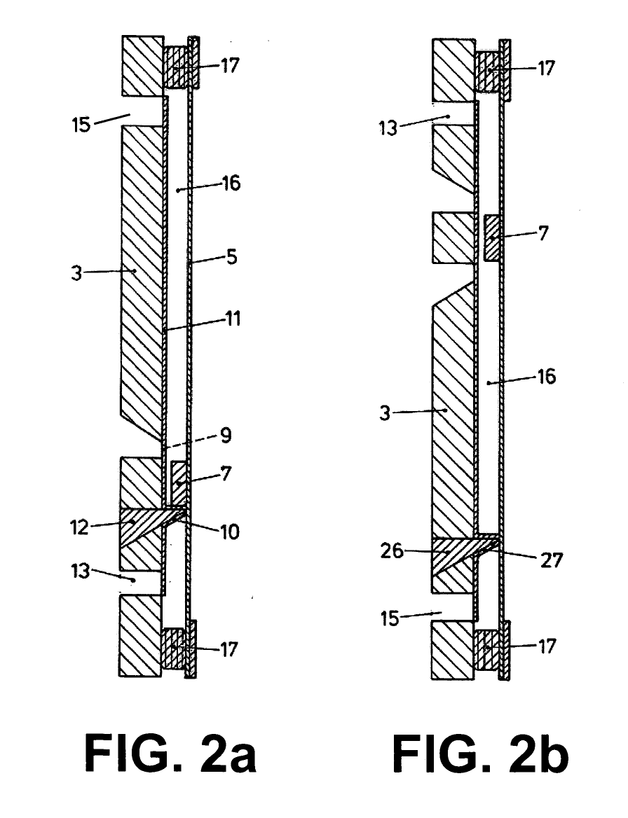

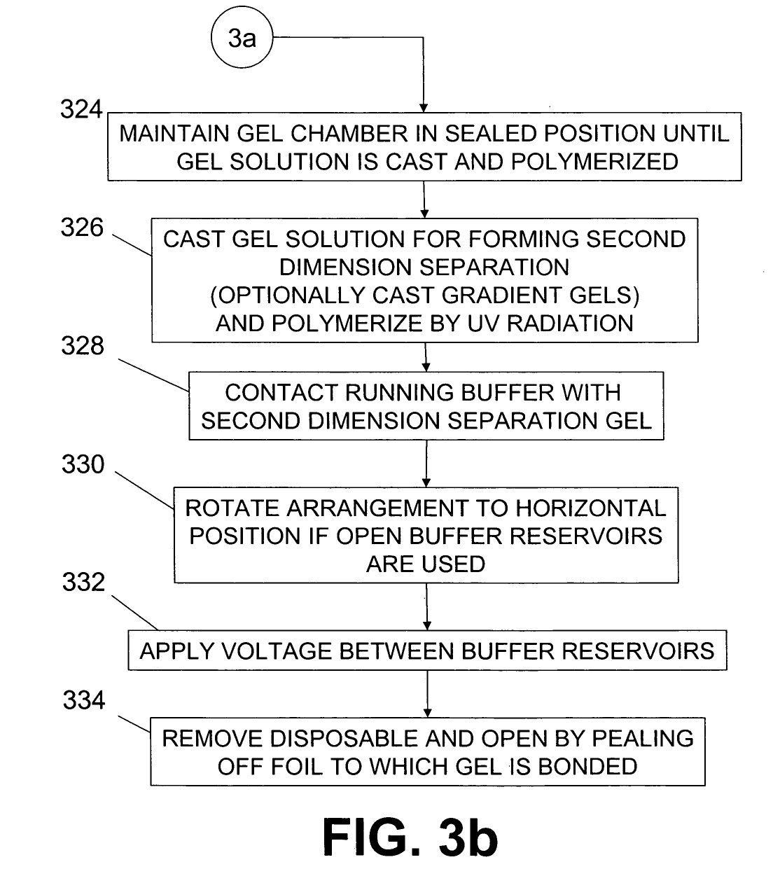

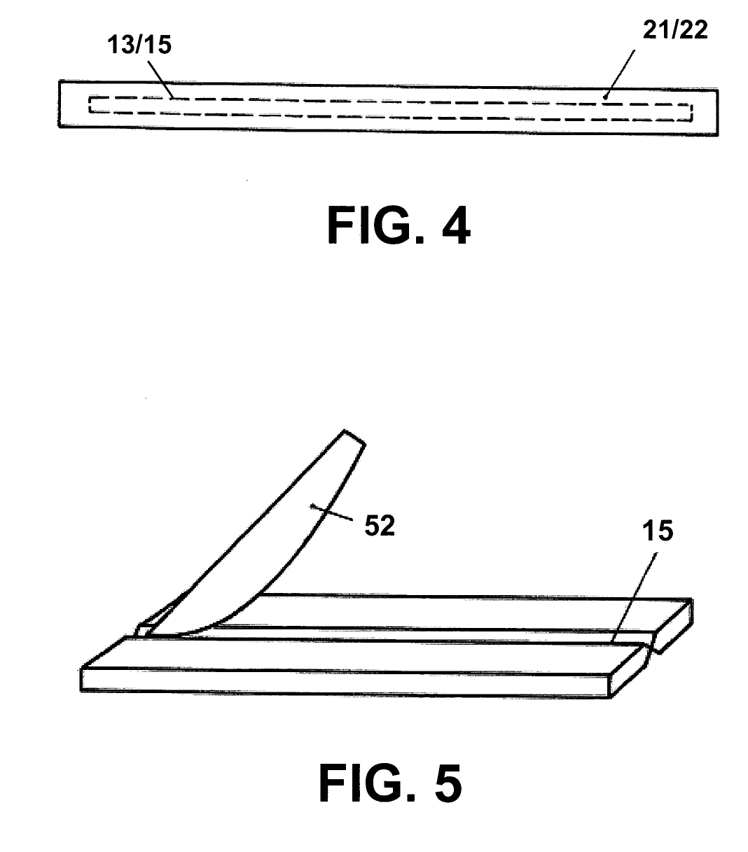

Another element applicable to the embodiments of the device 1 with valves, is the use of membranes or a blade at a gel/buffer interface 50. What is disclosed in the illustrative embodiment shown by FIG. 4 is the use of hydrophobic membranes, eventually supported, with the right combination of material, thickness and pore size, made such as, e.g. from PET, PE, PP or PES, with the function of acting as a barrier for the liquid gel solution during even vertical casting but allowing liquid and normal electrical contact between polymerized gel and SDS-containing buffer. In one embodiment, a working example is the Ultran® PES (Polyethersulfone) 5 KD membrane from Schleicher & Schuell GbmH (Einbeck, Germany)(Watman), normally used for filtration and biological applications, but in other embodiments, others can be employed as well, more or less efficiently depending also on the fact whether the gel contains SDS or not. In order to keep the disposable device 1 as simple as possible, in one embodiment slits or cavities 13 and 15 at the gel/buffer interface 50 are created after gel polymerization either in the form of longitudinal cut (shown by the dashed lines referred to by reference symbol 51 in FIG. 1b) made along a thinner integral lining of the molded disposable body 2, or by cutting the first elastic component layer 11 through the provided slits or cavities 13 and 15 in the hard component 3 of the body 2 by means of blades 52 (FIG. 5) integrated into the instrument 30. In an alternative embodiment, a simpler solution, when using flexible, reversible (i.e., any way) valves 9 and 10 to enclose the strip 7, is by means of the first elastic component layer 11, which is injection-molded together with the disposable body 2, closing the open edges of the gel chamber 16 during casting and polymerization, upon external actuation, in the same way as for closing the strip chamber 32 (FIG. 2b). In still another embodiment, another simple solution is to have a tape across the slits or cavities 13 and 15, which can be removed as soon as the contact to the buffer has to be established. In still another embodiment, the simplest of all solutions is to cast the gel in gel chamber 16 with the gel strip 7 by opening the inside valve 9 and leaving the outside valve 10 closed (FIG. 2a).

Process steps, which in one embodiment are automatically executed by the processing instrument 30, are described hereafter with reference to the attached figures.

First, in step 310 (FIG. 3a), and in reference to FIG. 1a, a disposable electrophoresis device is generally indicated by reference symbol 1. In one embodiment, a first gel strip 7 or a carrier 5 carrying the strip 7 is inserted into a disposable body 3. In another embodiment, step 310 is optional if the gel strip 7 and the carrier 5 are already integrated with disposable body 3. In still another alternative embodiment, the disposable 1 is configured such that an IEF strip, such as the first gel strip 7, is casted in situ with two valves 9 and 10 of the disposable body 3 arranged on both sides of the gel strip 7 being closed. The valves 9 and 10 may be actuated by inserts 12 and 14, which are part of an instrument 30 provided to process the disposable 1. In such an embodiment, the processing instrument 30 is programmed to automate the providing and casting of the IEF strip within the disposable 1. It is to be appreciated that the above insertion step may be carried out manually or by the processing instrument 30. With the disposable 1 provided in the processing instrument 30 as shown by FIG. 1a, the processing instrument 30 applies an external pressure 38 to the disposable 1 such that an elastic material 17 provided in the disposable 1 compresses to a distance a. In one embodiment, distance a is about 0.7 mm between the carrier 5 and the elastic component layer 11 covering an inner surface of the disposable body 3 of the disposable 1.

Second, in step 312, a protein sample (not shown) for separation is provided with a rehydration solution (not shown) to the disposable 1 with the two valves 9 and 10 at both sides of the gel strip 7 closed. It is to be appreciated that a strip chamber 32, which is defined between the closed valves 9 and 10, and which has one dimension being distance a, is filled with the protein sample/rehydration solution during this filling step.

Third, in step 314, a waiting period is executed for rehydration of the gel strip 7 within the strip chamber 32. In one embodiment, the rehydration time is at least one hour, and in other embodiments may be any time needed to rehydrate the material used for the gel strip 7. In addition, during rehydration, the temperature of a cooling block 34 of the processing instrument 30 provided adjacent the disposable device 1 is set at about 30° to about 35° Celsius.

Fourth, in step 316, IEF is run by applying ramping high voltage between the two opposed electrodes 36a and 36b either integrated or inserted at this moment by the processing instrument 30. During IEF, the instrument 30 controls also the temperature of the cooling block 34 which in one embodiment is set at about 20° Celsius.

Fifth, in step 318, distance a as shown in FIG. 1a is increased from about 0.7 mm between the foil 5 and the elastic component layer 11 of the disposable 1 to distance b, about 1.0 mm as shown in FIG. 1b, by releasing the instrument applied pressure from the device while stretching further the portions of the elastic component layer 11 functioning as valves 9 and 10 with the aid of the external inserts 12 and 14 to maintain the chamber of the strip 7 closed. The increase of the distance a according FIG. 1a, to distance b according to FIG. 1b is achieved as the integrated gasket 17, which is made out of an elastic soft component material, expands returning to its original shape. In the arrangement as shown in FIG. 1a the disposable body 3 is forced downwards towards the foil 5 by applying an external pressure, which is removed in the arrangement according to FIG. 1b.

Sixth, in step 320, the alkylation/SDS equilibration solutions are flown into the free space or channel 23 created on top of the strip 7 and empty at last. The free space or channel in one embodiment is about 0.3 mm.

Seventh, in step 322, the whole disposable 1 is rotated by about 90° to bring it in a more or less vertical position as shown in FIGS. 2a and 2b. This movement can be achieved upon rotation of the cooling block 34 (FIG. 1a) to which the disposable device 1 is steadily fixed by geometrical fitting and vacuum suction through the porous material as disclosed above.

FIG. 2a shows the design of the disposable as shown in FIG. 1b in vertical position with valve 10 on one side of the strips 7 still in closed position and valve 9 being opened.

Eighth, in step 324 (FIG. 3b), unless the slits or cavities 13 and 15 at the gel buffer interface have not yet been created or if they are closed by either the elastic component layer 11 or the additional membranes 21 and 22 as shown in FIG. 3, the valve 10 according to the embodiment of FIG. 2a with strip 7 at the bottom has to remain closed until the gel solution is cast and polymerized. As an alternative embodiment according to FIG. 2b with the gel strip 7 at the top an additional external valve 27 is arranged at the opposite side of the disposable 1. The spacing between foil 5 and cover plate 3 is still about 1 mm according to distance b of FIG. 1b.

Ninth, in step 326, the gel solution for forming the gel of the second dimension separation is provided through e.g. a hole (not shown) in the disposable body 3 for casting, thus achieving at the same time coupling with the strip 7. The gel solution is polymerized by a fast UV initiated reaction which is completed e.g. in less than 5 minutes. Polymerization is possible with the present invention due to the disposable body 3 (fully or partially) and layer 11 being transparent to UV radiation. If desired, gradient gels can be also cast in a similar manner.

Tenth, in step 328, valve 10 is opened within the arrangement of FIG. 2a, so that the running buffer can contact the gel for the second dimension separation. In an alternative embodiment, for the above purpose, the slits or cavities 13 and/or 15 are created if these were not yet present by for example, a blade function 32 as shown in FIG. 5. Another alternative embodiment is to proceed soon to the next step if the slits or cavities 13 and 15 were closed by additional membranes 21 and 22, respectively, as shown in FIG. 4.

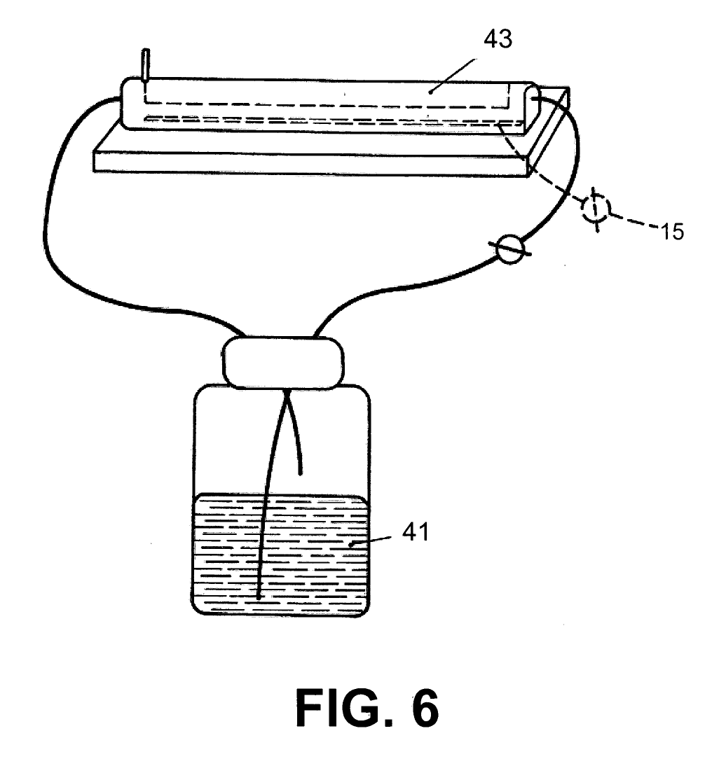

Eleventh, in step 330, eventually the arrangement is rotated back to a horizontal position as in FIG. 6, if open buffer reservoirs are used, otherwise this rotation is not absolutely necessary. If the reservoirs are part of the instrument, which means not integrated into the disposable, they can now be joined as shown with reference to FIG. 6 and designated with the reference numbers 41 and 42.

Twelfth, in step 332, the running buffer is introduced and the second dimension run is initiated by applying voltage to the electrodes (not shown) between the two reservoirs. The electrodes are either integrated into the disposable or in the instrument. In one embodiment, the second dimension run is conducted at a controlled temperature such as, for example, 20° Celsius. The running buffer is circulated as necessary.

Thirteenth, in step 334, after the second dimension run is completed, the disposable 1 is removed from the instrument and opened to remove the gel by pealing off the foil 5, to which the gel is bonded.

The above described process with reference to the attached drawings is of course an example suitable for describing the present invention and is not at all limiting to the present invention. The type of material used for producing the disposable device 1, including the elastic component layer 11 for the valves, the carrier 5, compressible or elastic parts 17, etc., could be changed in an appropriate manner. The use of a UV or light transparent material for the disposable device 1 is preferred so that UV or light initiated polymerisation of the second gel in the gel chamber 16 of the device 1 is possible, but it should not be a limiting factor to the present invention. Furthermore using two, three or more valves is possible. One feature of the present invention of course is, that the distance between the inner opposing surfaces of layer 11 and the carrier 5, on which the first gel strip 7 is attached is variable, which means that after the first dimension separation, the distance therebetween can be expanded, for example, due to the arranged compressible component or gasket 17.

The combination of at least one valve 9 or 10 around the gel strip 7 with the variable distance between the inner opposed surfaces of the carrier 5 and the elastic component layer 11 (e.g., about 0.7 mm and about 1.0 mm during first and second dimension respectively) raises the number of allowed positions for the at least one valve 9 or 10 from two to three and offers advantages compared to the prior art. First of all, no or minimum sample excess is required to rehydrate the gel strip 7 as the volume of the strip chamber 32 created by the valves 9 and 10 corresponds to the volume of the rehydrated strip. In this way, also IEF can be run under optimal conditions with no liquid excess on top of the gel strip 7. Space is created on top of the gel strip 7 only after IEF to introduce flowing equilibration solutions, thus providing also optimal equilibration conditions. Finally, as a thicker gel with a small space above the gel strip 7 is required to achieve proper coupling and perform a good second dimension analysis in terms of field homogeneity, 2D resolution and reproducibility, optimal conditions are provided also in this subsequent step.

Unlike the prior art, UV-initiated fast polymerization is adopted in the field of two-dimensional gel electrophoresis, choosing an initiator that is stable in the acrylamide gel solution until exposed to a light source whose wavelength range comprises its absorbance spectrum. Valves 9 and 10 are used only to close the gel strip 7 in a tight or closed environment, and not as barriers between the gel in the gel chamber 16 during the second dimension separation and the gel strip 7 in the strip chamber 32 because the gel can be polymerized quickly after the first dimension and doesn't have to be precast. Thus chemistry, storage time and conditions as well as waiting time for post- or pre-IEF polymerization are no longer an issue. Because polymerization proceeds fast in the present invention, such as from using the method disclosed in commonly assigned U.S. patent application Ser. No. 11/278,975, the disclosure of which is herein incorporated fully by reference, the use of a coupling gel such as, for example, agarose can also be eliminated. The gel solution can now fill completely the gel chamber 16, contacting, covering and enclosing the gel strip 7 therein, while this is not possible with the traditional method, making use of ammonium persulfate (APS) and N,N,N′,N′-tetramethylethylenediamine (TEMED) as initiator and catalyst respectively of radical polymerization. These reagents indeed have to be added and mixed at the last moment as they start immediately polymerization already during casting, thus causing already preparation problems, and because the reaction proceeds slowly taking normally more than one hour to be completed, loss of resolution obtained during the first dimension and diffusion of acrylamide monomers into the strip, causing possible cross-linking with the proteins, become other important issues. For the same reasons, fast UV polymerization becomes also particularly convenient when casting gradient gels.

Although the various embodiments of the present invention have been described using specific terms, such description is for illustrative purposes only, and it is to be understood that changes and variations may be made without departing from the spirit or scope of the following claims.

What is claimed is:

1. A disposable electrophoresis device for separation of a complex protein sample using two-dimensional gel electrophoresis, comprising:
a carrier with a first area for a first dimension gel strip and a second area for a second dimension gel where said two areas are directly in contact with each other; and
a body made of a hard component and a flexible component layer, said body being provided at a variable distance from the carrier, said body defining at least one cavity used for external actuation of at least one valve, wherein closing of said valve is represented by a reversible stretching of said flexible component layer in a direction towards the carrier upon external actuation of said valve through said cavity.

2. The device according to claim 1 wherein the body is at least partially UV transparent.

3. The device according to claim 1 wherein said carrier is a material selected from a polymer film, a foil, and a glass plate.

4. The device according to claim 1 wherein said carrier has gel-bond properties.

5. The device according to claim 1 wherein the flexible component layer is located at an inner surface of the body defining a bottom of the at least one cavity in the hard component.

6. The device according to claim 1 wherein the hard component comprises at least two cavities used for external actuation of the flexible component layer acting as two valves on both sides of the first area of the first dimension gel strip.

7. The device according to claim 1 wherein the variation of distance between the body and the carrier is obtained by means of at least one elastic component in sandwich arrangement between said body and said carrier.

8. The device according to claim 7 wherein the at least one elastic component is provided along a perimeter of the second area and is part of said device.

9. The device according to claim 1 wherein the hard component further comprises ports for sample loading, flowing of the equilibration solutions, casting of the gel solution or venting.

10. The device according to claim 1 wherein the hard component is a material selected from PMMA, PC, PE, PET, PP and COC.

11. The device according to claim 1 wherein the flexible component layer is a material selected from thermoplastic elastomers, PTS—Thermoflex®, Santoprene®, and other materials compatible with the hard component to enable component injection molding and to which the second dimension gel does not stick.

12. The device according to claim 1 wherein the device further comprises electrodes.

13. The device according to claim 1 wherein said device is configured to have said electrodes introduced externally.

14. The device according to claim 1 wherein the body further comprises cavities at an interface between the second dimension gel and a running buffer.

15. The device according to claim 14 wherein the cavities at the interface between the second dimension gel and the running buffer are sealed by additional membranes.

16. The device according to claim 15 wherein the additional membranes is a material having a thickness and a pore size that functions as a barrier for a liquid gel solution during casting of the second dimension gel in the second area but which allows liquid and normal electrical contact between a polymerized gel and a SDS-containing buffer.

17. The device according to claim 15 wherein the additional membranes is a material selected from PET, PE, PP and PES.

18. The device according to claim 14 wherein the cavities at the interface between the second dimension gel and the running buffer are being created only after gel polymerization by means of a blade function.

19. The device according to claim 1 wherein outer dimensions of said device are such to facilitate robotic handling.

20. The device according to claim 1 wherein the body further comprises cavities at the interface between the second dimension gel and a running buffer, and said device further comprises at least one external buffer reservoir from which the running buffer for the second dimension separation can be supplied to the device through said cavities.

21. The device according to claim 1 wherein the device is provided to, with an external side of the carrier contacting thereon, a cooling block through which a vacuum suction can be applied.

22. The device according to claim 21 wherein the cooling block is a material selected from a porous material, a ceramics, a metal, and other heat-conductive alloys.

23. A method for separation of a complex protein sample using two-dimensional gel electrophoresis within the device according to claim 1, said method comprising:
loading the sample in a rehydration solution onto the first dimension gel strip arranged on the carrier with valves closed on both sides of the first dimension gel strip with a distance between the carrier and the body corresponding approximately to a thickness of the first dimension gel strip rehydrated; and
creating a free space or channel above the first dimension gel strip to allow flowing of equilibration solutions after rehydration and separation in the first dimension by increasing the distance between the carrier and the body while stretching further down the valves at both sides of the first dimension gel strip to maintain a closed environment.

24. The method according to claim 23, further comprising:
introducing a gel solution used for a second dimension separation with at least one valve at one side of the first dimension gel strip open and the increased distance between the carrier and the body, wherein said gel solution couples with the first dimension gel strip at the same time; and
polymerizing the gel solution by using ultraviolet radiation.

25. The method according to claim 24, further comprising:
providing the device in a vertical position; and
casting of the gel solution to form the second dimension gel.

26. The method according to claim 25, further comprising:
separating from the device the carrier with the first dimension gel and second dimension gel attached.
